(19) United States
(12) Patent Application Publication
(10) Pub. No.: US 2015/0257676 A1
(43) Pub. Date: Sep. 17, 2015

(54) FLEXIBLE SAMPLING TUBE FOR PATIENT MONITORING

(71) Applicant: ORIDION MEDICAL 1987 Ltd., Jerusalem (IL)

(72) Inventors: Tim Fries, Louisville, CO (US); Erich Faust, Longmont, CO (US)

(73) Assignee: ORIDION MEDICAL 1987 Ltd., Jerusalem (IL)

(21) Appl. No.: 14/203,931

(22) Filed: Mar. 11, 2014

(51) Int. Cl. A61B 5/097; A61B 5/00; A61B 5/08; G01N 1/22; A61B 1/267

(52) U.S. Cl. CPC A61B 5/097; G01N 1/22; A61B 1/2676; A61B 5/082; A61B 5/6852

(57) ABSTRACT

A gas sampling device includes a connector configured to connect to a gas analyzing monitor. The gas sampling device further includes a first portion connected to the connector comprising a flexible tube for transmitting gas. The gas sampling device further includes a second portion connected to the first portion that includes a wand. The wand is configured for transmitting gas and is more rigid than the first portion. The gas sampling device further includes a rounded tip connected to the second portion. The rounded tip and second portion are configured to be inserted into a lung and transmit gas from inside the lung to the gas analyzing monitor.

### TECHNICAL FIELD

The present disclosure relates generally to sampling tubes, and more particularly to a system and device for gas sampling.

### BACKGROUND

Present gas sampling devices, including many end-tidal carbon dioxide (EtCO2) monitors, measure gas from a patient using an external mechanism. For example, many gas measuring devices connect to a patient using an external oral cannula, an external nasal cannula, an external oral/nasal cannula, or an external connection from an endotracheal tube. These devices are located at, or near, a patient's nose and mouth.

### SUMMARY

According to the present disclosure, disadvantages and problems associated with previous techniques for monitoring patients may be reduced or eliminated.

In certain embodiments, a gas sampling device includes a connector configured to connect to a gas analyzing monitor. The gas sampling device further includes a first portion connected to the connector comprising a flexible tube for transmitting gas. The gas sampling device further includes a second portion connected to the first portion that includes a wand. The wand is configured for transmitting gas and is more rigid than the first portion. The gas sampling device further includes a rounded tip connected to the second portion. The rounded tip and second portion are configured to be inserted into a lung and transmit gas from inside the lung to the gas analyzing monitor.

Certain embodiments of the present disclosure may provide one or more technical advantages. In conventional gas sampling systems, it is not possible to distinguish between gases transmitted from the individual lungs. In certain embodiments of this disclosure, an improved gas sampling device may be inserted into a patient's lungs to monitor gas directly from the lung. In certain embodiments, the gas sampling device has a semi-rigid wand that is designed to work with the flexible bronchoscope and allows the clinician to monitor EtCO2 at different spots within a lung. In certain embodiments, the clinician may monitor each side of the lungs individually using the gas sampling device. In these embodiments, the clinician may monitor an individual lung, which allows the clinician to identify a source of respiratory issues.

Certain embodiments of the present disclosure may include some, all, or none of the above advantages. One or more other technical advantages may be readily apparent to those skilled in the art from the figures, descriptions, and claims included herein. Moreover, while specific advantages have been enumerated above, various embodiments may include all, some, or none of the enumerated advantages.

### BRIEF DESCRIPTION OF THE DRAWINGS

For a more complete understanding of the present disclosure and it features and advantages, reference is now made to the following description, taken in conjunction with the accompanying drawings, in which:

FIG. 1 illustrates an example gas sampling device that includes a wand and rounded tip for insertion into a patient's lungs, according to certain embodiments of the present disclosure;

FIG. 2 illustrates an example gas sampling device inserted into a lung, according to certain embodiments of the present disclosure;

FIG. 3 illustrates two example gas sampling devices inserted into the lungs, according to certain embodiments of the present disclosure;

FIG. 4 illustrates a gas sampling system that includes two gas sampling devices and a gas sampling monitor, according to certain embodiments of the present disclosure; and

FIG. 5 illustrates an example method for implementing a gas sampling device, according to certain embodiments of the present disclosure.

### DETAILED DESCRIPTION

A capnograph measures air exhaled by patient through a tube. This tube may also be referred to as a gas sampling line or a gas sampling device as it is used in this disclosure. One end of the gas sampling device is connected to the patient through an external mechanism such as a mask, cannula, endotracheal tube, or an external connection to a respirator and/or ventilator. The other end of the gas sampling device is connected to a gas sampling monitor, in this particular example a capnograph. The complete gas sampling tube plus gas sampling monitor may be referred to as a gas sampling system.

Current gas sampling systems are limited because, as described above, the gas sampling tube is connected to a patient through an external mechanism. These external mechanisms do not allow a clinician to identify and detect certain respiratory issues that may exist in the internal airways and organs of a patient. In addition, current gas sampling and analysis systems have other limitations that may influence the gas analysis measurements, such as imperfect connections to a patient.

According to certain embodiments of the present disclosure, an improved gas sampling device is provided that addresses these problems with previous gas sampling devices. In these embodiments, the gas sampling device has a wand that is designed to work with a flexible bronchoscope and allow a clinician to monitor certain characteristics of a sampled gas, such as EtCO2, at particular spots within a lung. In certain other environments, the gas sampling device allows the clinician to monitor each side of the particular lung individually. In certain other embodiments of the present disclosure, a gas sampling system is provided that includes at least two gas sampling devices and a gas sampling monitor. In these embodiments, the gas sampling monitor is connected to the gas sampling devices. The gas sampling monitor is configured to receive transmitted gases from the two gas sampling devices analyze the transmitted gas to determine where one or more characteristics of the transmitted gases and to generate data for display indicative of the characteristics of the transmitted gases. In this example, the characteristics may include at least an EtCO2 measurement. Thus, the gas sampling system allows clinicians to monitor EtCO2 of the individual areas simultaneously, which allows the clinician to compare two EtCO2 measurements of the individual areas and potentially narrow down the source of respiratory issues.

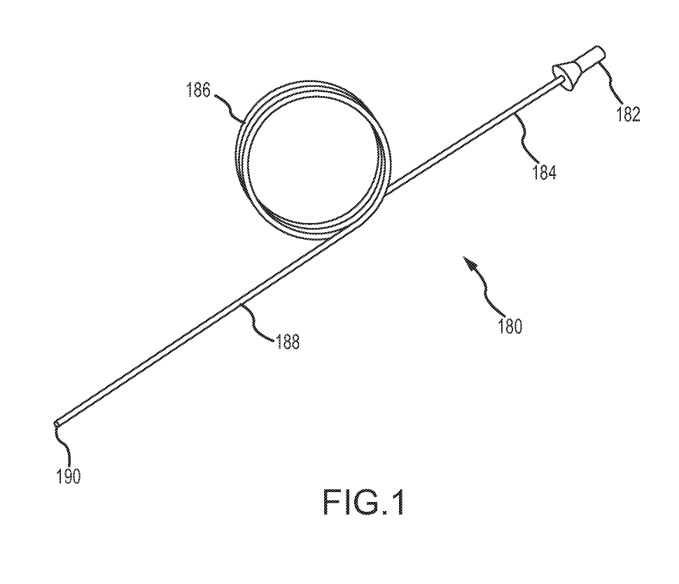

FIG. 1 illustrates an example gas sampling device 180 that includes a wand 188 and rounded tip 190 for insertion into a patient's lungs, according to certain embodiments of the present disclosure. In the illustrated embodiment, the gas sampling device 180 includes a connector 182, a filter 184, a tube 186, a wand 188, and a rounded tip 190. In the example, the filter 184 is connected to the tube 186, which is connected to the wand 188, which is connected to the rounded tip 190. Each of filter 184, tube 186, wand 188, and rounded tip 190 may be referred to as portions of gas sampling device 180. However, in certain other embodiments, each of these portions may be arranged and different configurations. For example the present disclosure contemplates that filter 184 may be arranged as connected to rounded tip 190. Thus, the present disclosure contemplates any suitable arrangement of these portions of gas sampling device 180.

Gas sampling device 180 may be configured to be inserted into a patient or a patient's airway in various configurations. For example, in certain embodiments, gas sampling device 180 may be inserted in the nose of a patient. As another example, gas sampling device 180 may be inserted in the mouth of a patient. As yet other example, gas sampling device 180 may be inserted during a tracheostomy. The present disclosure contemplates any other similar use of gas sampling device 180.

Gas sampling device 180 may be configured to receive gas from the patient and transmit the gas to a gas sampling monitor, such as a capnograph. In other suitable configurations, gas sampling device 180 may be configured to connect to any other suitable device or other connector. For example, gas sampling device 180 may be configured to connect to another gas sampling line, sensor, or any other suitable device.

Connector 182 may refer to any suitable connector for connecting gas sampling device 180 to a gas analyzing monitor. For example, connector 182 may refer to a luer connector.

Filter 184 may refer to any suitable filter. Filter 184 may refer to any filter that filters moisture, fluid, contaminants, or any other similar substance. For example filter 184 may comprise a hydrophilic wick.

Tube 186 may refer to any suitable tube for transmitting gas. For example in 186 may refer to a plastic tube that includes an inner wall where gas is transmitted from a patient to a gas sampling monitor. Tube 186 may be a flexible tube, in certain embodiments. In certain other embodiments, tube 186 or may be a rigid tube.

Wand 188 may refer to suitable device for insertion into a patient. For example, wand 188 may be inserted into the patient's airway. As another example, wand 188 may be inserted into the patient's lung.

Wand 188, according to certain embodiments, may refer to a semi-rigid tube that has sufficient flexibility to be inserted into a patient. According to these embodiments, the tube may be configured to transmit air from the patient's airway and/or the patient's lung. The semi-rigid tube may be 500 mm in length, according to one embodiment. According to certain embodiments, wand 188 may be more rigid than tube 186.

Rounded tip 190 may refer to any suitable tip to wand 188 to facilitate insertion of wand 188 into a patient and transmit gas to wand 188. For example, rounded tip 190 may comprise a soft rubber tip to wand 188 that would prevent any damage to a patient's airway and/or lungs during insertion. As another example, rounded tip 190 may comprise a bull nose tip to wand 188 to facilitate insertion into the patient and to prevent damage to a patient's airway and/or lungs during insertion.

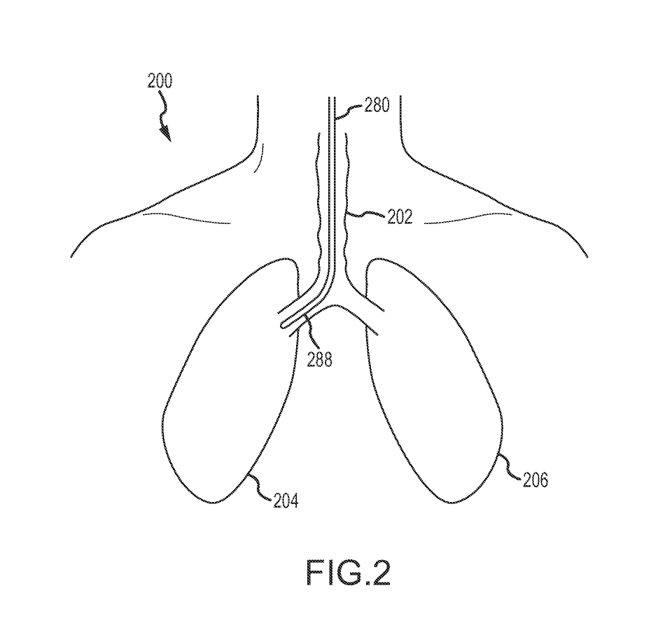

FIG. 2 illustrates example gas sampling device 280 inserted into a lung 204 of a patient 200, according to certain embodiments of the present disclosure. Gas sampling device 280 includes at least a wand 288. Gas sampling device 280 with wand 288 may be substantially similar to gas sampling device 180 with wand 188 of FIG. 1.

In the illustrated embodiment, gas sampling device 180 has been inserted through trachea 202 and inserted into lung 204. In certain embodiments, gas sampling device 180 may be coupled to a bronchoscope or other similar visualization device that facilitates insertion and placement of gas sampling device 180, wand 188, and rounded tip 190 into a patient. The bronchoscope may comprise either a flexible or a rigid bronchoscope. In certain environments of the disclosure, gas sampling device 180 may include any suitable connector to connect bronchoscope with gas sampling device 180 including clips, lumens, or any other suitable mechanism.

In certain embodiments, gas sampling device 280 may be inserted into a patient with assistance of a bronchoscope without coupling the bronchoscope to gas sampling device 280. In other embodiments, gas sampling device 280 may be inserted into a patient without the assistance of the bronchoscope.

Although the illustrated embodiment illustrates insertion of gas sampling device 280 into patient's lung 204, the present disclosure contemplates insertion of gas sampling device 180 into any other suitable airway or location inside a patient for purposes of gas sampling and analysis. For example, in certain other embodiments, gas sampling line 280 may be inserted into trachea 202, lung 206, or any other suitable location.

According to certain embodiments of the present disclosure, wand 288 is comprised of suitable material to be inserted into patient 200. For example, in the illustrated embodiment, wand 288 is rigid enough to be inserted through trachea 202 and flexible enough to be inserted around the patient's anatomy into lung 204. In certain embodiments, wand 288 may comprise plastic, rubber, any suitable natural or synthetic compounds, or any other suitable material. In certain environments, wand 288 may be reinforced on the outside (or external wall or external surface) of wand 288 or on the inside (or internal wall or inner surface) of wand 288 with any suitable material such as metal, rubber, any suitable natural or synthetic compounds.

According to certain embodiments of the present disclosure, gas sampling device 280 is inserted into lung 204 to allow the clinician to monitor EtCO2 of lung 204 directly. This allows the clinician to narrow down the source of respiratory issues if they exist in lung 204. In other embodiments, gas sampling device 280 with wand 288 may be placed within lung 204 to sample and receive gas certain areas within lung 204 or any other suitable location.

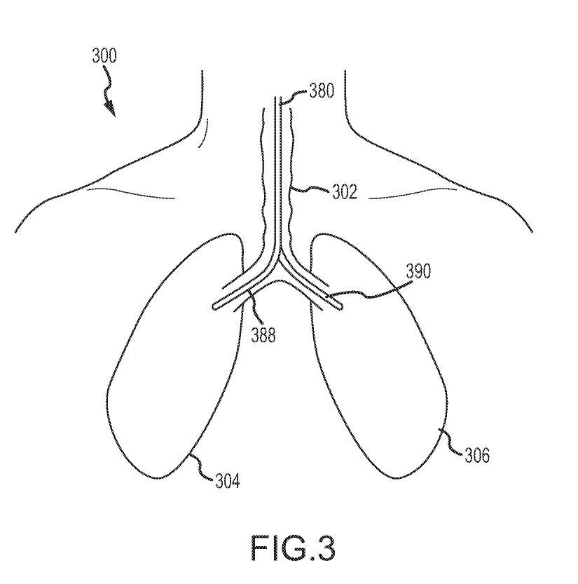

FIG. 3 illustrates example gas sampling device 380 inserted into a lung 304 and lung 306 of a patient 300, according to certain embodiments of the present disclosure. As shown in FIG. 3, gas sampling device 380 includes a branch that includes at two wands including wand 388 and wand 390. Although FIG. 3 illustrates two wands, the present disclosure contemplates any suitable number of wands. Wand 388 and 390 may be substantially similar to wand 188 of FIG. 1 and gas sampling device 380 may be substantially similar to gas sampling device 180 of FIG. 1.

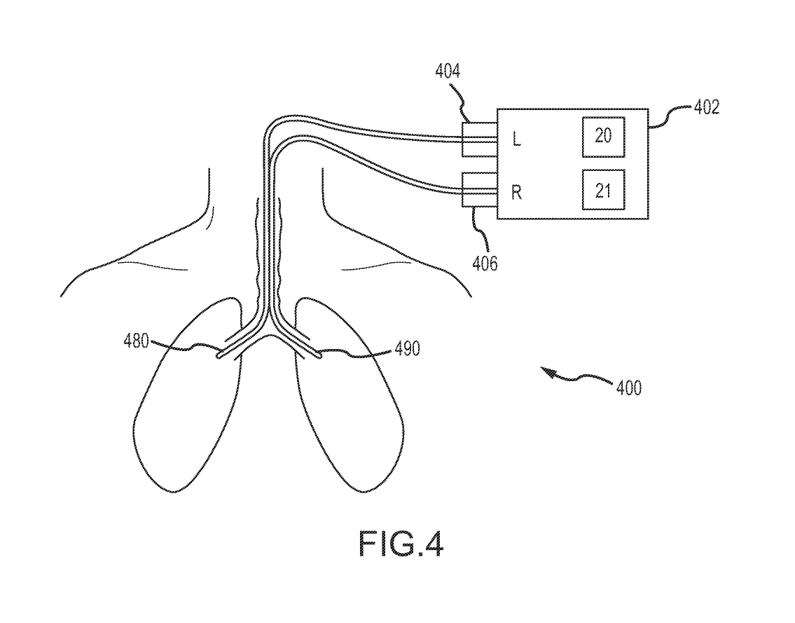

According to certain embodiments of the present disclosure gas sampling device 380 includes a branch with wands 388 and 390 to sample and receive gas from lungs 304 and 306 directly and simultaneously. In addition to the branch illustrated in FIG. 3, the present disclosure contemplates utilizing two or more separate gas sampling devices, such as gas sampling device 180, to monitor several areas of a patient. For example, as disclosure below with reference to FIG. 4, at least two gas sampling devices may be used to simultaneously sample and receive gas from two different areas of a patient and transmit the gas to a gas sampling monitor.

FIG. 4 illustrates a gas sampling system 400 that includes two gas sampling devices 480 and 490 and a gas sampling monitor 402, according to certain embodiments of the present disclosure. In the illustrated embodiment, gas sampling device 480 is connected to monitor 402 with connector 404 and gas sampling device 490 is connected to monitor 402 with connector 406.

According to certain embodiments of the present disclosure gas sampling system 400 facilitates sampling, transmission, and analysis of gas samples directly from certain areas internal to a patient and facilitates comparison of the areas. For example, according to the illustrated embodiment, gas sampling device 480 may provide a first transmitted gas to monitor 403 for first lung and gas sampling device 490 may provide a second transmitted gas from a second lung. As shown in the illustrated embodiment the two samples may be analyzed by monitor 402 to determine one or more characteristics of the first transmitted gas and the second transmitted gas. In the example, monitor 402 may generate data for display indicative of the one or more characteristics of the first transmitted gas and second transmitted gas.

According to certain embodiments of the present disclosure monitor 402 may display the characteristics of the transmitted gas as coming from a left lung and a right lung. In certain other embodiments contemplated by the disclosure, monitor 402 may display any other suitable parameters to display and distinguish between the areas of gas sampled by gas sampling devices 488 and 490.

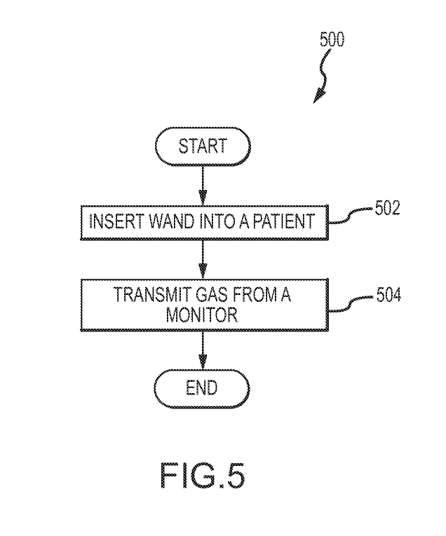

FIG. 5 illustrates an example method 500 for implementing a gas sampling device, according to certain embodiments of the present disclosure. Method 500 begins at step 502 where a gas sampling device including a wand is inserted into a patient. For example, the wand may be inserted into the patient's airway. As another example, the wand may be inserted into the patient's lung. In certain embodiments, at least a portion of the gas sampling device may be inserted with the help of a bronchoscope as described above with reference to FIG. 2.

The wand may refer to a semi-rigid tube that has sufficient flexibility to be inserted into a patient. According to these embodiments, the tube may be configured to transmit air from the patient's airway and/or the patient's lung. The semi-rigid tube may be 500 mm in length, according to one embodiment. According to certain embodiments, the wand may be more rigid than other portions of the gas sampling device.

Next, at step 502, gas is transmitted from gas sampling device to a gas sampling monitor. In certain embodiments, the gas sampling monitor may include a capnograph. In certain other embodiments, the gas sampling monitor may include a multiparameter monitoring device that monitors gas along with other patient parameters such as oxygen saturation. Transmission of sampled gas from the gas sampling device to the gas sampling monitor allows a clinician to monitor certain characteristics of a sampled gas, such as EtCO2, at particular spots within a lung. In certain embodiments, the gas sampling device allows the clinician to monitor each side of the particular lung individually. In other embodiments, at least two gas sampling devices are inserted into a patient and connected to the gas sampling monitor. In these embodiments, the at least two gas sampling devices may be inserted individually or may be branched from a single line or tube.

In certain embodiments, the gas sampling monitor may be configured to receive transmitted gases simultaneously from the two gas sampling devices and analyze the transmitted gas to determine where one or more characteristics of the transmitted gases and to generate data for display indicative of the characteristics of the transmitted gases. In this example, the characteristics may include at least an EtCO2 measurement. Thus, the gas sampling system allows clinicians to monitor EtCO2 of the individual lungs at the same time, which allows the clinician to compare two EtCO2 measurements of the individual lungs and potentially narrow down the source of respiratory issues.

Although this disclosure has been described in terms of certain embodiments, alterations and permutations of the embodiments will be apparent to those skilled in the art. Accordingly, the above description of the embodiments does not constrain this disclosure. Other changes, substitutions, and alterations are possible without departing from the spirit and scope of this disclosure, as defined by the following claims.

What is claimed is:

1. A gas sampling device, comprising:
a connector configured to connect to a gas analyzing monitor;
a first portion connected to the connector comprising a flexible tube for transmitting gas;
a second portion connected to the first portion comprising a wand, the wand configured for transmitting gas and being more rigid than the first portion; and
a rounded tip connected to the second portion, the rounded tip and second portion configured to be inserted into a lung and transmit gas from inside the lung to the gas analyzing monitor.

2. The gas sampling device of claim 1, wherein the second portion comprises a 500 mm semi-rigid plastic tube.

3. The gas sampling device of claim 1, wherein the first portion comprises a filter.

4. The gas sampling device of claim 1, wherein the rounded tip comprises a bull nose tip.

5. The gas sampling device of claim 1, wherein at least the second portion is configured to be coupled to a bronchoscope to facilitate placement of the rounded tip.

6. A gas sampling system, comprising:
a first gas sampling device comprising at least a first rounded tip and a first wand configured to be inserted into a first lung;
a second gas sampling device comprising at least a second rounded tip and a second wand configured to be inserted into a second lung; and
a gas sampling monitor configured to:
connect to the first and second gas sampling device;
receive at least a first transmitted gas from the first sampling device;
receive at least a second transmitted gas from the second sampling device;
analyze at least the first and second transmitted gas to determine one or more characteristics of the first transmitted gas and the second transmitted gas; and
generate data for display indicative of the one or more characteristics of the first transmitted gas and the second transmitted gas.

7. The gas sampling system of claim 1, wherein the first gas sampling device comprises a 500 mm semi-rigid plastic tube.

8. The gas sampling system of claim 1, wherein the first gas sampling device comprises a filter.

9. The gas sampling system of claim 1, wherein the first rounded tip comprises a bull nose tip.

10. The gas sampling system of claim 1, wherein at least the first gas sampling device is configured to be coupled to a bronchoscope to facilitate placement of the first rounded tip.
